(19) United States
(12) Patent Application Publication
(10) Pub. No.: US 2014/0081262 A1
(43) Pub. Date: Mar. 20, 2014

(54) NEARFIELD ULTRASOUND ECHOGRAPHY MAPPING

(71) Applicant: Boston Scientific Scimed Inc., Maple Grove, MN (US)

(72) Inventors: Josef V. Koblish, Sunnyvale, CA (US); David L. McGee, San Miguel, CA (US)

(73) Assignee: Boston Scientific Scimed Inc., Maple Grove, MN (US)

(21) Appl. No.: 14/031,518

(22) Filed: Sep. 19, 2013

Related U.S. Application Data
(60) Provisional application No. 61/703,344, filed on Sep. 20, 2012.

(51) Int. Cl. A61B 18/14

(52) U.S. Cl. CPC A61B 18/1492
USPC 606/41

(57) ABSTRACT

Various embodiments concern delivering an ablation therapy to different areas of the cardiac tissue and, for each of the areas, sensing an ultrasound signal with at least one ultrasound sensor, the ultrasound signal responsive to the ultrasound energy reflected from the area of cardiac tissue. Such embodiments can further include for each of the plurality of different areas of the cardiac tissue, associating with each area an indication of the degree to which the area of cardiac tissue was lesioned by the delivery of the ablation therapy based on the ultrasound signal and representing a map of the different areas on a display. A user input can select one of the different areas and the indication associated with the selected one area can be represented on the map.

### CROSS-REFERENCE TO RELATED APPLICATION

This application claims priority to Provisional Application No. 61/703,344, filed Sep. 20, 2012, which is herein incorporated by reference in its entirety.

### TECHNICAL FIELD

The present disclosure relates generally to analyzing anatomical structures within the body. More specifically, the present disclosure relates to devices, systems, and methods for characterizing the condition of tissue as part of a cardiac map.

### BACKGROUND

In ablation therapy, it is often necessary to determine various characteristics of body tissue at a target ablation site within the body. In interventional cardiac electrophysiology (EP) procedures, for example, it is often necessary for the physician to determine the condition of cardiac tissue at a target ablation site in or near the heart. During some EP procedures, the physician may deliver a mapping catheter through a main vein or artery into an interior region of the heart to be treated. Using the mapping catheter, the physician may then determine the source of a cardiac rhythm disturbance or abnormality by placing a number of mapping elements carried by the catheter into contact with the adjacent cardiac tissue and then operating the catheter to generate an electrophysiology map of the interior region of the heart. Once a map of the heart is generated, the physician may then advance an ablation catheter into the heart, and position an ablation electrode carried by the catheter tip near the targeted cardiac tissue to ablate the tissue and form a lesion, thereby treating the cardiac rhythm disturbance or abnormality. In some techniques, the ablation catheter itself may include a number of mapping electrodes, allowing the same device to be used for both mapping and ablation.

Various ultrasound-based imaging catheters and probes have been developed for visualizing body tissue in applications such as interventional cardiology, interventional radiology, and electrophysiology. For interventional cardiac electrophysiology procedures, for example, ultrasound imaging devices have been developed that permit the visualization of anatomical structures of the heart directly and in real-time. In some electrophysiology procedures, for example, ultrasound catheters may be used to image the intra-atrial septum, to guide transseptal crossing of the atrial septum, to locate and image the pulmonary veins, and to monitor the atrial chambers of the heart for signs of a perforation and pericardial effusion.

### SUMMARY

The present disclosure relates to devices, systems, and methods for characterizing tissue properties using ultrasonic echography.

In example 1, a system for characterizing the condition of multiple areas of cardiac tissue of a heart comprises a catheter configured to be introduced into the heart, the catheter comprising: at least one electrical sensor, the at least one electrical sensor configured to sense an electrical signal from the surface of the cardiac tissue; at least one ultrasound transducer, the at least one ultrasound transducer configured to receive ultrasound energy reflected from the cardiac tissue and generate a signal indicative of the intensity of the reflected ultrasound energy; and an ablation element, the ablation element configured to deliver an ablation therapy to the cardiac tissue. Example 1 further includes a user interface comprising a display and a user input. Example 1 further includes memory and control circuitry configure to determine, for each of a plurality of different areas of cardiac tissue, an indicator of the degree to which the area of cardiac tissue was lesioned by delivery of the ablation therapy based on the ultrasound signal, save the indicators for the plurality of different areas of cardiac tissue as respectively associated with the plurality of different areas of cardiac tissue in memory, generate a map on the display representing the plurality of different areas of the cardiac tissue based on the electrical signal, receive a selection of one or more of the plurality of different areas of the cardiac tissue from the user input, and represent the indication associated with each of the selected one or more areas of cardiac tissue on the map based on the selection.

In example 2, the system of example 1, wherein the indicators indicate the depth of lesioning through the cardiac tissue.

In example 3, the system of either of examples 1 or 2, wherein the control circuitry is configured to determine the depth of lesioning through the cardiac tissue based on the intensity of ultrasound energy reflected by tissue of the area at different depths.

In example 4, the system of any of examples 1-3, wherein the control circuitry is configured to determine, for each of the plurality of different areas, the degree to which the area was lesioned by calculating a change in a level of intensity of the ultrasound reflected from the area, the change calculated from a first ultrasound scan performed before delivery of the ablation therapy to the area and a second ultrasound scan performed following delivery of at least a portion of the ablation therapy to the area.

In example 5, the system of any of examples 1-4, wherein the control circuitry is configured to represent the indication associated with each of the selected one or more areas of cardiac tissue by showing, on or alongside the map on the display, a graphic representation of tissue thickness and the depth of the lesion through the tissue thickness.

In example 6, the system of any of examples 1-5, wherein the control circuitry is configured to represent the indicator associated with each of the selected one or more areas of cardiac tissue by showing, on or alongside the map on the display, a profile of ultrasound reflectivity of the area of cardiac tissue.

In example 7, the system of any of examples 1-6, wherein the control circuitry is configured to represent the indication associated with each of the selected one or more areas of cardiac tissue in a manner that indicates whether the selected one or more areas of cardiac tissue was transmurally lesioned by the delivery of the ablation therapy.

In example 8, the system of any of examples 1-7, wherein the control circuitry is configured to represent only one of the indicators of the degree to which the plurality of different areas of the cardiac tissue were lesioned at a time based on the user input.

In example 9, the system of any of examples 1-8, wherein the at least one ultrasound transducer comprises at least three ultrasound transducers, the at least three ultrasound transducer positioned on different portions of the catheter to respectively scan different fields; and the control circuitry is configured to determine the orientation of the catheter with respect to the cardiac tissue based on with which of the at least three ultrasound transducers the cardiac tissue can be detected to be proximate to the at least three ultrasound transducers and with which of the at least three ultrasound transducers the cardiac tissue cannot be detected to be proximate to the at least three ultrasound transducers.

In example 10, the system of any of examples 1-9, wherein the control circuitry is configured to represent the orientation of the catheter in relationship to the map generated on the display.

In example 11, the system of any of examples 1-10, wherein the control circuitry is configured to determine whether the plurality of different areas of cardiac tissue form a contiguous series and highlight one or more areas of the map where the contiguous series is not lesioned.

In example 12, the system of any of examples 1-11, wherein the control circuitry is configured to move a curser generated on the display; and the selection of the one or more of the plurality of different areas of the cardiac tissue is based on the curser being moved onto the one or more of the plurality of different areas on the map.

In example 13, the system of any of examples 1-12, wherein the user input is controlled based on the movement of the catheter in the heart; and the selection of the one or more of the plurality of different areas of the cardiac tissue is based on the catheter being moved onto the one or more of the plurality of different areas of the cardiac tissue in the heart.

The example 14, the system of any of examples 1-13, further comprising a positional sensor on the catheter, the positional sensor configured to output a signal indicative of the spatial position of the catheter within the heart, wherein the control circuitry is configured to generate the map based on the signal and the electrical cardiac signal.

In example 15, a method for representing information characterizing the condition of multiple areas of cardiac tissue of a heart comprises sensing an electrical signal from the surface of the cardiac tissue with one or more electrodes on a catheter; delivering an ablation therapy to a plurality of different areas of the cardiac tissue; for each of the plurality of different areas of the cardiac tissue, sensing an ultrasound signal with at least one ultrasound sensor within the heart, the ultrasound signal responsive to the ultrasound energy reflected from the area of cardiac tissue; for each of the plurality of different areas of the cardiac tissue, associating with the area an indication of the degree to which the area of cardiac tissue was lesioned by the delivery of the ablation therapy based on the ultrasound signal; representing a map of the plurality of different areas on a display, the map based at least in part of the electrical signal; receiving a user input selecting one of the plurality of different areas; and representing the indication associated with the selected one area based on the user input.

In example 16, the method of example 15, wherein the indications indicate the depth of lesioning through the cardiac tissue.

In example 17, the method of either of examples 15 or 16, further comprising determining, for each of the plurality of different areas, the degree to which the area was lesioned by calculating a change in a level of intensity of the ultrasound reflected from the area, the change calculated from a first ultrasound scan performed before delivery of the ablation therapy to the area and a second ultrasound scan performed following delivery of at least a portion of the ablation therapy to the area.

In example 18, the method of any of examples 15-17, wherein the at least one ultrasound sensor comprises at least three ultrasound sensors, the at least three ultrasound sensors positioned on different portions of the catheter to respectively scan different fields; and the method further comprises determining the orientation of the catheter with respect to the cardiac tissue based on with which of the at least three ultrasound sensors the cardiac tissue can be detected to be proximate to the at least three ultrasound sensors and with which of the at least three ultrasound sensors the cardiac tissue cannot be detected to be proximate to the at least three ultrasound sensors.

In example 19, a system for characterizing the condition of multiple areas of tissue, the system comprising a catheter, the catheter comprising at least one ultrasound sensor, the at least one ultrasound transducer configured to receive ultrasound energy reflected from the cardiac tissue and generate a signal indicative of the intensity of the reflected ultrasound energy; and an ablation element on the catheter, the ablation element configured to deliver an ablation therapy to the cardiac tissue. Example 19 further comprises a user interface and control circuitry configure to determine, for each of a plurality of different areas of cardiac tissue, an indicator of the depth of lesioning through the cardiac tissue of the area by delivery of the ablation therapy based on the ultrasound signal and generate a map on the user interface representing the indicators of the depth of lesioning in respective association with the plurality of different areas of the cardiac tissue.

In example 20, the system of example 19, wherein the control circuitry is configured to determine the depth of lesioning, for each of the plurality of different areas of cardiac tissue, through the cardiac tissue based on the intensity of ultrasound energy reflected by cardiac tissue of the area at different depths.

While multiple embodiments are disclosed, still other embodiments of the present invention will become apparent to those skilled in the art from the following detailed description, which shows and describes various illustrative embodiments of the present disclosure. Accordingly, the drawings and detailed description are to be regarded as illustrative in nature and not restrictive.

### BRIEF DESCRIPTION OF THE DRAWINGS

FIG. 1 shows an exemplary system for characterizing cardiac tissue in accordance with various aspects of this disclosure;

FIG. 2 shows a block diagram of components for characterizing cardiac tissue in accordance with various aspects of this disclosure;

FIGS. 3A-F show a map for characterizing cardiac tissue in accordance with various aspects of this disclosure; and

FIG. 4 shows a flowchart of a method for characterizing cardiac tissue and controlling an ablation therapy in accordance with various aspects of this disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Various cardiac abnormalities can be attributed to improper electrical activity of cardiac tissue. Such improper electrical activity can include, but is not limited to, generation of electrical signals, conduction of electrical signals, and/or mechanical contraction of the tissue in a manner that does not support efficient and/or effective cardiac function. For example, an area of cardiac tissue may become electrically active prematurely or otherwise out of synchrony during the cardiac cycle, thereby causing the cardiac cells of the area and/or adjacent areas to contract out of rhythm. The result is an abnormal cardiac contraction that is not timed for optimal cardiac output. In some cases, an area of cardiac tissue may provide a faulty electrical pathway (e.g., a short circuit) that causes an arrhythmia, such as atrial fibrillation or supraventricular tachycardia. In some cases, inactivate tissue (e.g., scar tissue) may be preferable to malfunctioning cardiac tissue.

Cardiac ablation is a procedure by which cardiac tissue is treated to inactivate the tissue. The tissue targeted for ablation may be associated with improper electrical activity, as described above. Cardiac ablation can lesion the tissue and prevent the tissue from improperly generating or conducting electrical signals. For example, a line, a circle, or other formation of lesioned cardiac tissue can block the propagation of errant electrical signals. In some cases, cardiac ablation is intended to cause the death of cardiac tissue and to have scar tissue reform over the lesion, where the scar tissue is not associated with the improper electrical activity. Lesioning therapies include electrical ablation, radiofrequency ablation, cyroablation, microwave ablation, laser ablation, and surgical ablation, among others.

Ideally, the ablation therapy can be delivered in a minimally invasive manner, such as with a catheter introduced to the heart through a vessel, rather than surgically opening the heart for direct access (e.g., as in a maze procedure). For example, a single catheter can be used to perform an electrophysiology study of the inner surfaces of a heart to identify electrical activation patterns. From these patterns, a clinician can identify areas of inappropriate electrical activity and ablate cardiac tissue in a manner to kill or isolate the tissue associated with the inappropriate electrical activation. However, the lack of direct access in a catheter-based procedure may require that the clinician only interact with the cardiac tissue through a signal catheter and keep track of all of the information that the catheter collects or is otherwise associated with the procedure. In particular, it can be challenging to keep track of the areas that are targeted for ablation, the condition of ablated areas, and the progress in creating a pattern of ablated areas that kills or isolates improperly activating tissue.

Moreover, ablating tissue in a pattern composed of multiple tissue sites to isolate improperly activating tissue can be complicated by difficulty in determining which areas of the tissue were fully inactivated (e.g., by a transmural lesion). Conventionally, an ablation treatment may be considered successful if the electrophysiology catheter no longer senses improper electrical activity from a particular section of tissue following lesioning. However, the lesioned tissue may merely be stunned or temporarily non-conductive. It may be difficult to distinguish between fully ablated tissue with no conduction and tissue that is rendered temporarily nonconductive due to edema. In these cases, the cessation of improper electrical activity may only be temporary, and the improper electrical activity may return later. Edema, for example, can temporarily block improper electrical activity following lesioning, where the improper electrical activity resumes once the edema subsides. In some cases, a whole line or other pattern of ablated tissue can be thwarted by a small amount of tissue along the line recovering from the delivery of ablation therapy to once again conduct unwanted electrical signals. Excessive treatment of the tissue, on the other hand, may risk the ablation of more tissue than intended and consequently inactivating more tissue than intended, possibly degrading output capabilities.

The present disclosure concerns, among other things, methods, devices, and systems for tracking the state of ablated tissue in a map. For example, various embodiments concern generating a map identifying a plurality of different areas of a cardiac tissue that have been treated with an ablation therapy and presenting indicators, based on ultrasound signals, characterizing the degree to which selected area were ablated. While normal cardiac tissue, partially ablated tissue, and tissue with edema may all have the same or similar levels of electrical activation, the ultrasound reflectivity properties of these tissues can be different. The present disclosure discusses exploiting the variability in the ultrasound reflectivity properties of these tissues to generate a map indicating the cardiac areas that have been fully ablated (e.g., with a transmural lesion) and the areas that are not fully ablated and may support the redevelopment of unwanted electrical signals in the heart.

Information regarding the condition of cardiac tissue can be used to determine whether the tissue is healthy, whether the tissue should be lesioned (e.g., for the first time or an additional time), and/or whether the tissue was successfully ablated in a previous treatment, among other things. As discussed herein, this information can be collected and saved in memory for retrieval, the information being displayed based on selection of a marker corresponding to the area of cardiac tissue. In this way, a clinician can retrieve information characterizing the degree to which tissue was lesioned for each of a plurality of different areas to which ablation therapy was delivered. This information, and the manner of retrieval of the information, can be useful for the clinician in determining whether any area of a line or other pattern of ablated tissue is not contiguous with fully lesioned tissue. For example, a plurality of different areas of tissue can be highlighted (e.g., by color or shading) on a map to show whether a recently delivered ablation therapy formed a contiguous series of durable lesions (e.g., transmural lesions) before the ablation procedure is ended and the catheter is withdrawn. In some cases, a series of markers respectively associated with different areas of a conduction block can be represented as a linear line if the different areas are associated with contiguous lesions, while a broken and/or non-linear line can represent a series of non-contiguous lesions. A further example concerns the use of this information after arrhythmia has returned, where a clinician can use the manner of data retrieval and presentation as discussed herein to evaluate which particular area of a conduction block may be allowing the propagation of unwanted electrical signals (e.g., a shallower lesion and/or deeper tissue for a particular area of the conduction block line allowing arrhythmia redevelopment). As such, a weak link in a conduction block can be identified. This tissue can then be retargeted for further ablation and the rest of the tissue can be spared further ablation.

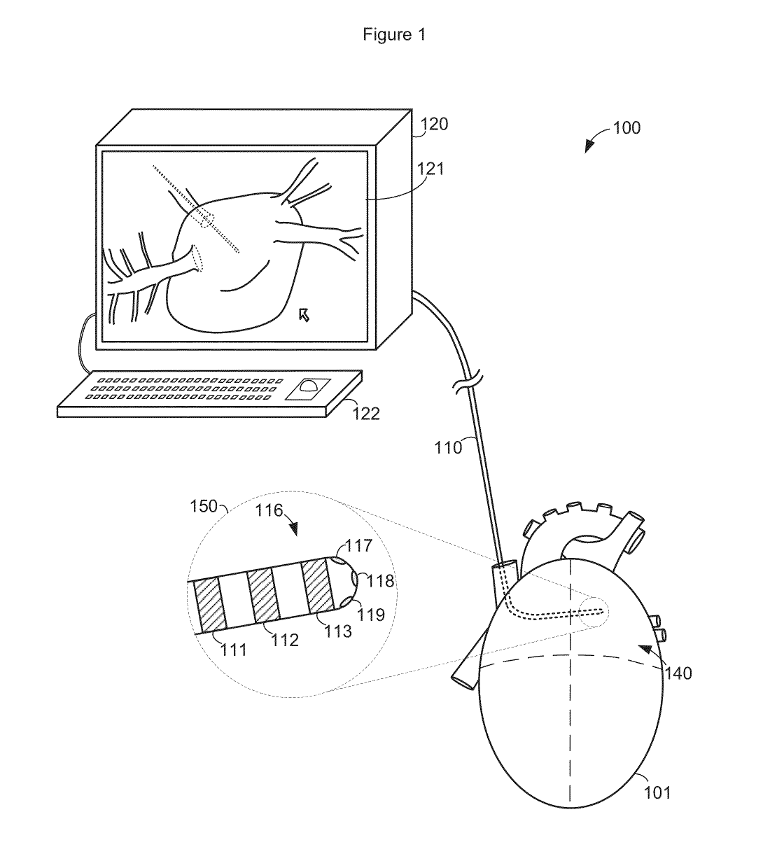

FIG. 1 is an illustrative embodiment of a system 100 for mapping the ablation of cardiac tissue. The system 100 includes a catheter 110 connected to a control unit 120. The catheter 110 can comprise an elongated tubular member having a distal end 116 configured to be introduced within a heart 101 or other area of the body. As shown in FIG. 1, the distal end 116 of the catheter 110 is within the left atrium 140.

As shown in the window 150 of FIG. 1, the distal end 116 of the catheter 110 includes electrodes 111-113. The electrodes 111-113 can be configured for sensing signals, such as electrical cardiac signals. The electrodes 111-113 can additionally or alternatively be used to deliver ablative energy to cardiac tissue. Although three electrodes are illustrated in FIG. 1, various embodiments can have a lesser or greater number of electrodes. Furthermore, electrodes in various other embodiments can be multi-functional (e.g., sensing cardiac signals and delivering ablation therapy) or can have dedicated functionality (e.g., sensing or ablation only).

The distal end 116 of the catheter 110 can further include ultrasound transducers 117-119. The ultrasound transducers can be used for characterizing cardiac tissue, as will be discussed further herein. Ultrasound transducers 117-119 can send ultrasound waves in a pulsing mode and receive ultrasound waves reflected from tissue in a sensing mode. When excited electrically in a pulsing mode, the ultrasound transducers can create pressure waves which travel into the surrounding environment. In the sensing mode, the ultrasound transducers can produce an electrical signal as a result of receiving acoustic waves reflected back to the ultrasound transducers from tissue, which can be processed and displayed on the display 121 of the control unit 120. In various embodiments, an ultrasound sensor is configured to deliver acoustic waves at a frequency greater than about 20 MHz (e.g., in a near field application) from the distal tip of the catheter 110. Ultrasound transducers can be mounted on the exterior of the catheter 110 or may be housed within the body of the catheter 110, where the ultrasound waves are sent and received through the housing of the catheter 110. Each ultrasound transducer can have multi-functionality (e.g., sending and sensing ultrasound energy) in some embodiments while each ultrasound transducer in some other embodiments may have dedicated functionality (e.g., transmitting or sensing ultrasound energy). In various embodiments, the ultrasound transducers comprise piezoelectric elements formed of a polymer such as PVDF or a piezoceramic material such as PZT. Although three ultrasound transducers are illustrated in FIG. 1, various embodiments can have a lesser or greater number of ultrasound transducers, such as three ultrasound transducers arrayed around the circumference of the distal end of the catheter 110 and an additional ultrasound transducer on the distal tip facing distally. In various embodiments, ultrasound transducers are arranged in a phased array on the distal end 116 of the catheter 110. In some embodiments, a single rotating ultrasound transducer may be provided inside the catheter 110 to scan an area of tissue, although multiple rotating ultrasound transducers can also be provided.

In various embodiments, ultrasound transducers 117 and 119 and another ultrasound transducer on the other side of the catheter can be arrayed around the circumference of the catheter 110. For example, a plurality of ultrasound transducers can be circumferentially arrayed around the perimeter of the catheter 100, each ultrasound transducer facing a different direction. The direction that an ultrasound transducer faces can correspond to the area that the ultrasound transducer scans. For example, a first ultrasound transducer can be positioned on the catheter 110 to send ultrasonic pulse waves in a first direction projected from the catheter 110 and/or receive ultrasonic pulse waves from the first direction, a second ultrasound transducer can be positioned on the catheter 110 to send ultrasonic pulse waves in a second direction projected from the catheter 110 and/or receive ultrasonic pulse waves from the second direction, and a third ultrasound transducer can be positioned on the catheter 110 to send ultrasonic pulse waves in a particular direction projected from the catheter and/or receive ultrasonic pulse waves from the third direction, wherein the first, second, and third directions are all different relative to one another and/or cover different fields relative to the catheter 110. A greater or lesser number of ultrasound transducers can be arranged in this manner. One or more of the ultrasound transducers can be positioned to also send ultrasonic pulse waves distally of the catheter 110 and/or receive ultrasonic pulse waves distally from the catheter 110 (e.g., ultrasound transducer 118). In various embodiments, the orientation of the catheter 110 can be determined based on which of these ultrasound transducers can be used to detect tissue proximate the ultrasound sensor and which of the ultrasound transducers cannot detect tissue proximate the ultrasound sensor, as will be described further herein. In various embodiments, the orientation of the distal end 116 of the catheter 110 can be determined based on which of these ultrasound transducers can be used to detect tissue in contact with the distal end 116 of the catheter 110 and which of the ultrasound transducers cannot detect tissue in contact with the distal end 116 of the catheter 110. In some cases, the catheter 110 is in a preferred orientation for delivering ablation therapy when the target tissue is in contact with the distal end 116 of the catheter 110.

The system 100 is capable acquiring and processing ultrasound signals in multiple modes simultaneously or sequentially. Ultrasound modes include M-mode, and/or A-Mode, for example. An ultrasound echography system operating in an M-mode can render moving two dimensional images of tissue in a sectional view. An ultrasound echography system operating in A-mode can show the amplitude of the received ultrasound energy, which can be used for determining the depth of tissue, characterizing one or more properties (e.g., density) of the tissue, and/or determining the proximity of the distal to tip to the tissue (e.g., for contact sensing).

The catheter 110 can include one or more lumens having conductors and/or other elements facilitating the transmission of signals, fluids, etc. along the catheter 110. Other members can also be moved through the catheter 110 within the one or more lumens, such as a guidewire or tendon for articulating the distal end 116. The catheter 110 can be articulated to aid in navigation and moving along various sections of cardiac tissue. For example, a tendon within a lumen of the catheter 110, and connected to a distal portion of the catheter 110, may be pulled in tension from the proximal end of the catheter 110 to bend a section of the catheter 110. A knob on a handle (not illustrated) of the catheter 110 may be used to create tension or slack in the tendon. One or more guide catheters (not illustrated) may also be used to support the catheter 110 in straightening and/or bending. The catheter 100 can be connected to one or more extensions proximally for bridging to the control unit 120. In various embodiments, the catheter 110 is open irrigated and includes one or more irrigation holes. Various other embodiments concern a non-irrigated catheter 110.

The control unit 120 of the system 100 includes a display 121 (e.g., LCD) for displaying information. The control unit 120 further includes a user input 122 which can comprise one or more buttons, toggles, a track ball, a mouse, touchpad, or the like for receiving user input. The control unit 120 can comprise a hardware console and software system for collecting and processing information as discussed herein for characterizing and mapping tissue. The control unit 120 can contain control circuitry for performing the functions described herein.

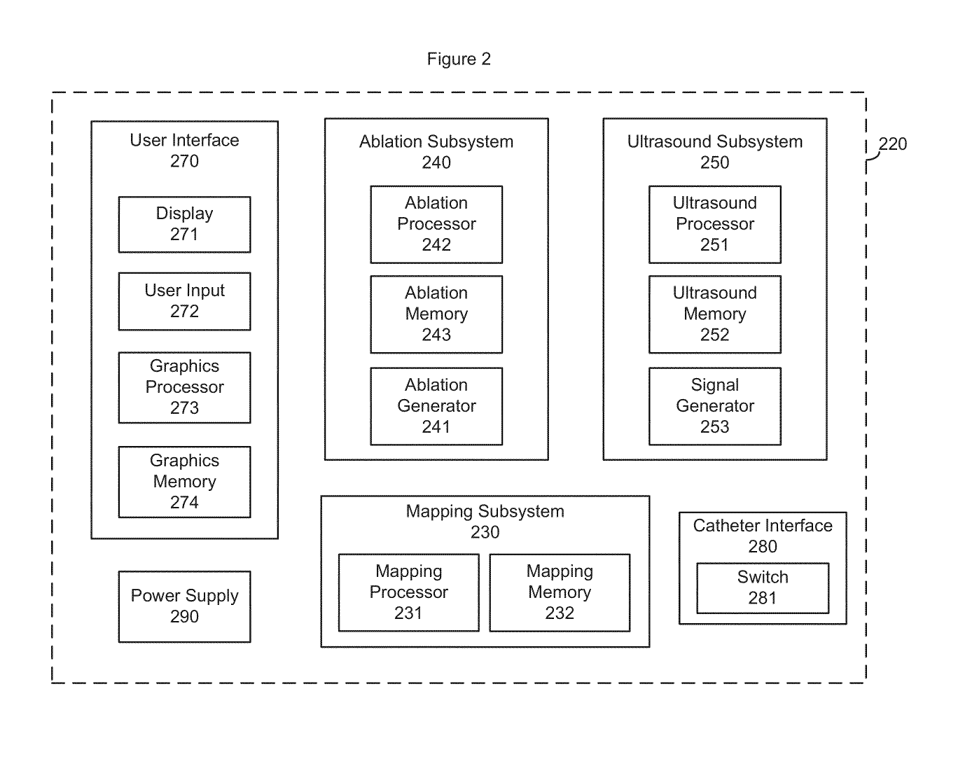

FIG. 2 illustrates a block diagram showing control circuitry and other components for performing functions described herein. The control circuitry can be housed within control unit 220, which can comprise a single housing or multiple housings among which components are distributed. The components of the control unit 220 can be powered by a power supply 290 which can supply electrical power to any of the components of the control unit 220 and the system 100. The power supply 290 can plug into an electrical outlet and/or provide power from a battery, among other options.

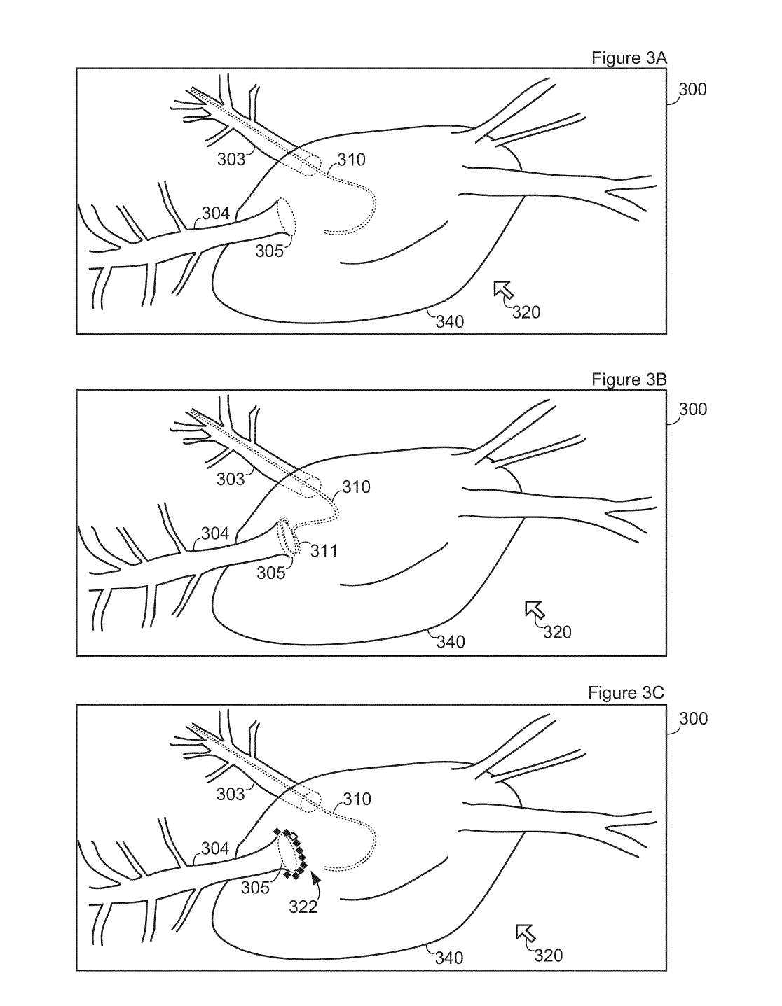

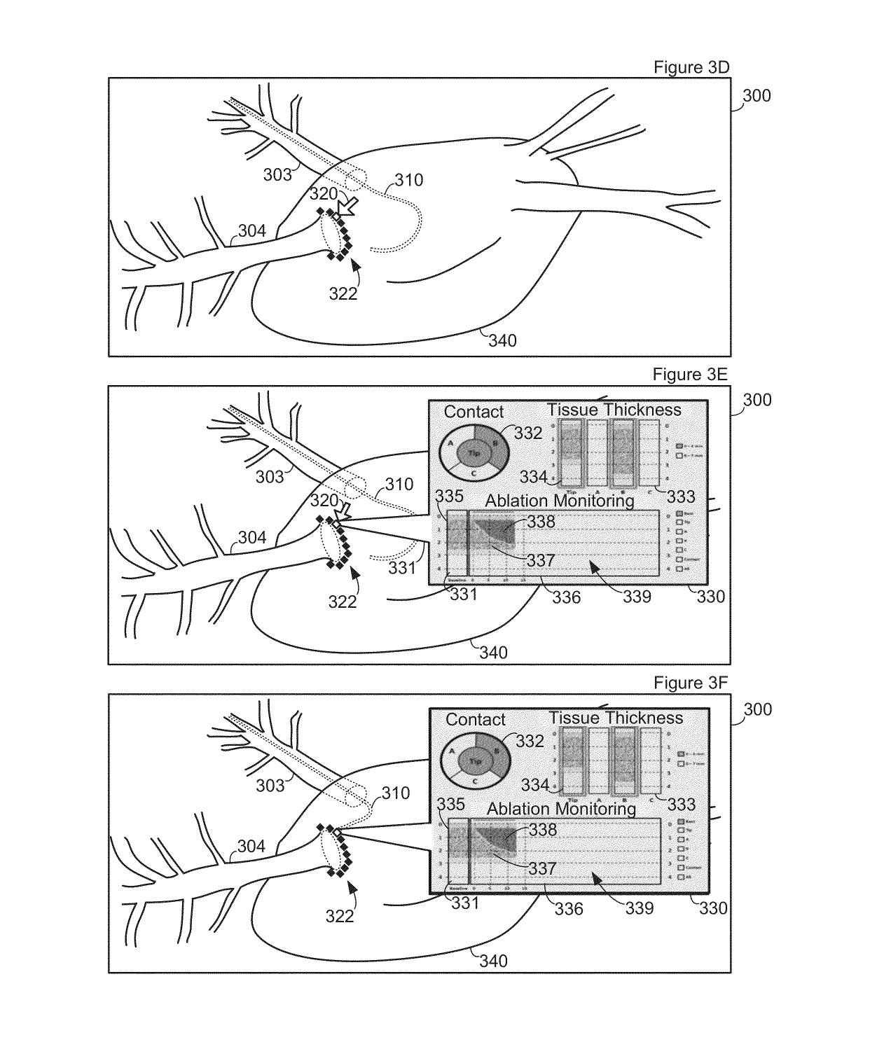

The block diagram of FIG. 2 illustrates a mapping subsystem 230 which includes components for operating the mapping functions of the system. The mapping functions can include sensing one or more cardiac signals from the surface of the heart (e.g., via electrodes 111-113 coupled with one or more conductors within the catheter 110), mapping conduction patterns, identifying unwanted electrical activity, and identifying one or more target sites within the heart, among other things. Target sites can include sections of cardiac tissue that support aberrant conductive pathways in the heart or are associated with improper cardiac function. The mapping processor 231 can be configured to execute program instructions stored in the mapping memory 232 to derive activation times and voltage distribution from the electrical signals obtained from the electrodes 111-113 to identify irregular electrical signals within the heart and/or perform other functions. The cardiac information can then be graphically displayed as a map on the display 271, such as the map shown in FIGS. 1 and 3A-F. An example mapping system that can be employed to detect electrical signals in myocardial tissue for use in identifying target treatment sites and/or for providing ablation energy to target sites is further described in U.S. Pat. No. 7,720,420, which is expressly incorporated herein by reference in its entirety for all purposes. Further details regarding electrophysiology mapping are provided, for example, in U.S. Pat. Nos. 5,485,849, 5,494,042, 5,833,621, and 6,101,409, each of which is expressly incorporated herein by reference in its entirety for all purposes.

In some embodiments, three dimensional mapping functions can be used to track the three dimensional position of the catheter 110. The electrodes 111-113 can be used to make impedance measurements to determine the three dimensional position of the catheter 110 in the cardiac space. Magnetic fields can additionally or alternatively be created and sensed by a sensor within the catheter 110 to determine the three dimensional position of the catheter 110 in the cardiac space. For example, the sensor can be sensitive to magnetic fields and can output a signal indicative of positional changes due to moving between different magnetic fields. The changes in the signal can be compared with the created magnetic fields to determine the location and/or movement of the sensor. The mapping subsystem 230 or other circuitry can support these functions. The three dimensional position of the catheter 110 can be used to determine from where along the heart particular electrical cardiac signals were sensed (e.g., the position of an electrode in the cardiac space at the moment a signal was sensed), for generating a graphical representation of the heart or other structures, for determining the location in the cardiac space of tissue associated with unwanted electrical activity (e.g., a target area), and/or determining to which areas of the heart ablation therapy is delivered, among other things.

The block diagram of FIG. 2 illustrates an ablation subsystem 240 which includes components for operating the ablation functions of the system. The ablation subsystem 240 includes an ablation generator 241. The ablation generator 241 can provide different therapeutic outputs depending on the particular configuration. For example, in the case of radiofrequency ablation, the ablation generator 241 can generate a high frequency alternating current signal to be output through one or more electrodes (e.g., electrodes 111-113), where ablative heat is generated upon application to tissue. Providing ablation energy to target sites is further described, for example, in U.S. Pat. No. 5,383,874 and U.S. Pat. No. 7,720,420, each of which is expressly incorporated herein by reference in its entireties for all purposes. In some other embodiments, the ablation generator 241 can generate microwave energy to be transmitted by a catheter to ablate targeted tissue or a solution that cools to cryoablate the targeted tissue. The ablation generator 241 may support any other type of ablation therapy. The ablation subsystem 240 may include an ablation processor 242 and ablation memory 243 for controlling ablation functions. For example, the ablation memory 243 can contain program instructions executable by the ablation processor 242 for controlling ablation functions as described herein, such as for managing the delivery of ablation energy.

The block diagram further illustrates an ultrasound subsystem 250 which includes components for operating the ultrasound functions of the system. The ultrasound subsystem 250 can include a signal generator 253 configured to generate a signal for ultrasound transmission. For example, the signal generator 253 may generate a signal (e.g., a 20 MHz signal) for transmission along a conductor of the catheter 110 to one or more of the ultrasound transducers 117-119 which can emit ultrasound waves based on the signal. The ultrasound subsystem 250 can include signal processing circuitry (e.g., a high pass filter) configured to filter and process reflected ultrasound signals as received by an ultrasound transducer in a sense mode and conducted to the ultrasound subsystem 250 through a conductor in the catheter 110. Filtering and processing may include filtering out noise frequencies and amplifying the signal among other functions for highlighting and identifying features of the signals indicative of particular tissue characteristics. The ultrasound subsystem 250 may comprise an ultrasound processor 251. The ultrasound processor 251 may perform signal processing functions, as well as perform other functions. For example, the ultrasound memory 252 can contain program instructions executable by the ultrasound processor 251 for performing the functions described herein, including measuring the intensity of reflected ultrasound energy and determining the degree to which cardiac tissue was lesioned by ablation therapy based on changes in the intensity of the reflected ultrasound energy. As discussed herein, the system may operate in an M-mode, an A-mode, and/or any other modes.

The block diagram further illustrates a user interface subsystem 270 which can support user input and output functionality. A display 271 (e.g., a liquid crystal display based screen) can be used to display any map, curser, catheter, target area, indication, determination, chart, plot, and/or any other information. A graphics processor 273 and graphics memory 274 may be used to support the display 271 functionality, and may be part of the display 271. A user input 272 can be used to allow a user to input information and make selections, among other things. For example, the user input 272 can allow a clinician to move a curser around a map generated on the display 271 by the graphics processor 273 executing instructions from the graphics memory 274 to make selections, such as selecting a particular area of cardiac tissue. User input 272 can log key and/or other input entries and route the entries to other circuitry. User input 272 may comprise a mouse, trackball, touchpad, touch screen, joystick, slider bar, or any other control.

A catheter interface 280 can provide a port for connecting the catheter 110 to the control circuitry of the control unit 220. A switch 281 can be used to selectively route signals to and from the different components of the control unit 220 along the conductors of the catheter 110.

Although the block diagram of FIG. 2 illustrates multiple processors and memory units, one or more processors can be used to implement the functions described herein. For example, a single processor could perform the functions of multiple subsystems, and as such the subsystems may share control circuitry. Although different subsystems are presented herein, circuitry may be divided between greater or lesser numbers of subsystems, which may be housed separately or together. In various embodiments, circuitry is not distributed between subsystems, but rather is provided as a unified computing system. Whether distributed or unified, the components can be electrically connected to coordinate and share resources to carry out functions.

FIGS. 3A-F illustrate a contrived map 300 in a chronological series demonstrating various mapping and tissue characterization features of the present disclosure. The map 300 can be generated by control circuitry and displayed on a display in connection with an ablation procedure employing ultrasound echography to evaluate the degree to which tissue was lesioned. FIG. 3A shows a map 300 of a portion 340 of the heart. The portion 340 of the heart can be, for example, the left atrium. A significant number of arrhythmia, such as atrial fibrillation, arise from the left atrium. In some cases, arrhythmia has been known to arise from the tissue surrounding the openings to the pulmonary veins in the left atrium. FIGS. 3A-F show a procedure attempting to isolate these tissues by forming a ring of ablated tissue around an opening 305 to a pulmonary vein 304.

FIG. 3A shows that a catheter 310 has been introduced into the portion 340 of the heart by a vessel 303. The catheter 310 could correspond to the catheter 110 of FIG. 1. An electroanatomical map of a cardiac structure (in this case the portion 340 of the heart) may be generated by moving electrodes of the catheter 310 along the inner surfaces of the cardiac tissue and sensing electrical cardiac activity. The electrodes may be moved along the inner surfaces by advancing and retracting the catheter 310 as well as by articulating the distal end of the catheter 310. Three dimensional positional information may further be collected to determine the locations (e.g., in three dimensional cardiac space) of the various cardiac areas from which the electrical cardiac activity is sensed. Tissue associated with arrhythmia may be identified based on the sensed electrical cardiac activity and the position of the tissue may be located based on the three dimensional information. Specifically, the activation times and voltages of specific sections of cardiac tissue may be compared with an overall cardiac rhythm and/or depolarization wave to identify tissue that is activating prematurely or otherwise out of rhythm with the rest of the chamber and/or heart. In this example, premature electrical activations can be identified from the sensed cardiac signal, the premature electrical activations being detected as occurring before adjacent tissue activated and/or before activations of the rest of the cardiac cycle. The location of this tissue may be identified based on the three dimensional location of the electrode that sensed the premature electrical activations at the time that the premature electrical activations were sensed. The location of the catheter 310 can be depicted on the map 300 in real time based on the detected three dimensional position of the catheter 310.

In the example of FIGS. 3A-F, an atrial arrhythmia may be identified based on errant electrical activations originating from around the opening 305 of the pulmonary vein 304, the errant electrical activations arising for one or more cardiac cycles before these sections of tissue should be activated according to the overall cardiac rhythm of the heart and/or before adjacent tissue activates. Based on the identification of tissue around the opening 305 of the pulmonary vein 304, these areas can be targeted for ablation. These areas can also be marked with markers on the display to indicate that these areas are associated with irregular electrical activation and/or are targeted for ablation. The identification of these areas may be done by a clinician and/or by control circuitry on the basis that they are each associated with irregular electrical cardiac activity.

FIG. 3B illustrates a partial ring 311 being formed out of the distal end of the catheter 310. The partial ring 311 can be formed by articulation of the distal end of the catheter 310 or the partial ring 311 shape can be the biased shape of the distal end, where an outer guide catheter is used to straighten the catheter 310 or form other shapes. The partial ring 311 shape can array the electrodes of the catheter 310 around the opening 305 of the pulmonary vein 304 to deliver ablation therapy to a plurality of different sections of tissue around the opening 305. As such, a plurality of lesions can be created by the catheter 310. Although the example of FIG. 3B shows a ring being formed to isolate and/or destroy tissue generating aberrant electrical signals around the opening 305 of the pulmonary vein 304, different shapes and/or different areas of cardiac tissue can be targeted to address various arrhythmias.

Before, during, and/or after the delivery of the ablation therapy, ultrasound scans can be made of the areas to which ablation therapy was targeted and/or delivered. Such ultrasound scans can including pulsing ultrasound energy to each of the areas and receiving ultrasound energy reflected from the areas. In various embodiments, each of the areas are scanned individually in a serial manner, as the limits of near field ultrasound require that the ultrasound transducer be very close to the area being scanned.

Measuring the intensity of the reflected ultrasound waves can provide information regarding characteristics of tissue, such as the density, contractility, and/or dynamic mobility of the tissue. For example, denser tissue will typically reflect more ultrasound energy than similar but less dense tissue. In some cases, lesioned tissue is denser than unlesioned tissue. As such, the density of cardiac tissue can be used as an indicator of the state of the tissue. An ultrasound sensor can measure more intense ultrasound energy reflected from denser sections of tissue and relatively less intense ultrasound energy reflected from less dense sections of tissue. For these cases, greater levels of ultrasound energy reflected from cardiac tissue indicates a lesion while lesser levels of ultrasound energy reflected from cardiac tissue indicates no lesion. A comparison can be made between the intensity levels of ultrasound energy measured before and after ablation therapy is delivered to determine whether the intensity level of ultrasound energy being reflected changes in association with the delivery of the ablation therapy. An increase in reflected ultrasound energy from an area of cardiac tissue following ablation therapy delivery can indicate the formation of a lesion from the ablation therapy while no increase in reflected ultrasound energy from the area of cardiac tissue following ablation therapy delivery can indicate that no lesion was formed from the ablation therapy. Typically, lesioned tissue is less contractile than unlesioned tissue. A comparison can be made between the contractility of tissue before, after, and/or during lesioning based on ultrasound energy measured before, during, and/or and after ablation therapy to determine whether the tissue is any less contractile in association with the delivery of the ablation therapy. A decrease in the contractility of an area of cardiac tissue following ablation therapy delivery can indicate the formation of a lesion from the ablation therapy while no change in the contractility of the tissue can indicate that no lesion was formed from the ablation therapy. Typically, lesioned tissue has less dynamic mobility than unlesioned tissue. A comparison can be made between the dynamic mobility of tissue before, after, and/or during lesioning based on ultrasound energy measured before, during, and/or and after ablation therapy to determine whether the tissue is any less dynamically mobile in association with the delivery of the ablation therapy. A decrease in the dynamic mobility of an area of cardiac tissue following ablation therapy delivery can indicate the formation of a lesion from the ablation therapy while no change in the dynamic mobility of the tissue can indicate that no lesion was formed from the ablation therapy.

A parameter of the reflected ultrasound energy can be measured to determine the degree to which a particular area of cardiac tissue was lesioned by the delivery of the ablation therapy. For example, a parameter indicative of the intensity of reflected ultrasound energy (e.g., amplitude) can be compared between two or more ultrasound scans of the area, where a first scan is performed before the delivery of the ablation therapy (e.g., as a baseline scan) and a second scan is performed during and/or after the delivery of the ablation therapy. If the parameter indicates a change in the reflectivity of ultrasound energy from the tissue, then a lesion can be determined to have been formed. A predetermined threshold representing a lesion can be set, whereby a change in intensity of ultrasound energy before and after ablation delivery can be compared to the threshold to determine whether the tissue was lesioned (e.g., a change greater than the threshold indicates a lesion while a change less than the threshold indicates no lesion). The scan can also determine the reflectivity of tissue at different tissue depths, and a comparison to the predetermined threshold can be performed for each of a plurality of depth ranges for each area of cardiac tissue. In various cases, a transmural lesion is desired for each of the targeted areas because the transmural lesion is the least likely to later resume generating and/or propagating unwanted electrical cardiac signals. As such, by determining the depth of a lesion based on the ultrasound reflectivity of tissue at different depths, it can be determined the degree to which an area of tissue was lesioned by the delivery of ablation therapy. The information indicating the degree to which an area of tissue was lesioned can be saved in memory for later retrieval and use, as will be further discussed herein.

FIG. 3C illustrates a plurality of markers 322 appearing on the screen. Each of the markers 322 represents an area of cardiac tissue that was targeted for lesioning and to which ablation therapy was delivered. As described herein, each area could be identified based on an electrical cardiac signal indicative of aberrant electrical activity and located in three dimensional space. These areas can then be indicated on a map generated on a display with the plurality of markers 322. As shown in FIG. 3C, the plurality of markers 322 form a partial ring around the opening 305 of the pulmonary vein 304. Each of these markers 322 can be associated with ultrasound information collected during and/or after the delivery of ablation therapy to the area, the ultrasound information indicative of the degree to which the area of tissue was lesioned.

The markers 322 can also be in a pattern of a conduction block, the markers 322 representing a contiguous series of cardiac tissue areas. The markers 322 can be displayed to indicate whether the represented tissue areas are lesioned. For example, the degree of lesioning of each of the tissue areas can be determined as described herein and each of the markers can be colored, shaded, shaped, or otherwise displayed in some manner to indicate whether tissue area respectively associated with the markers 322 is lesioned. A first color of marker may be used to represent lesioned tissue while a second color of marker may be used to represent unlesioned tissue. A series of markers of the first color can represent a contiguous series of lesioned tissue areas. However, if any of the markers in the series is of the second color, then this indicates that the lesioning is not contiguous. A single area of unlesioned tissue in a series can allow aberrant electrical signals to propagate past an otherwise successful conduction block. If a particular marker is of the second color (or otherwise indicated to not be fully lesioned), then the clinician can further investigate the tissue area and collected information as further shown herein.

FIG. 3D illustrates the curser 320 being used to select one of the markers 322. Curser 320 can be moved around the map 300 by a user input (e.g., by a touchpad). Any one of the markers 322 can be selected by a user moving the curser 320 over the marker, as is shown in FIG. 3D. In this and/or in other ways, any of the plurality of areas targeted for ablation therapy and/or to which ablation therapy was delivered can be selected. Although the areas of cardiac tissue are individually selected in the embodiment of FIGS. 3A-F by moving a curser 320 over one of a plurality of markers 322, areas of cardiac tissue can be individually selected in various other ways. For example, a particular marker may not be used to indicate targeted or ablated tissue, and moving a curser 320 over the tissue on the map may select the tissue. In any case, selection of cardiac tissue can trigger the display of further information in relation to the map 300 based on the selection, as shown in FIG. 3E.

FIG. 3E illustrates the display of a chart 330 of ultrasound information indicating the degree to which the area of cardiac tissue was lesioned, the area of cardiac tissue corresponding to the marker selected by the curser 320. The chart 330 includes a plurality of plots, each plot characterizing a different aspect of the cardiac tissue. Parts of this annotating information characterizes various aspects of the lesioning of the area of cardiac tissue to indicate the degree to which the area of tissue was lesioned by the delivery of the ablation therapy based on ultrasound information collected from the tissue before, during, and/or after the delivery of the ablation therapy.

Chart 330 includes a tissue depth plot 333. The tissue depth plot 333 shows the thickness of cardiac tissue as respectively measured with by the ultrasound transducers of the catheter 310. For example, column 334 represents the thickness of cardiac tissue as detected by the tip ultrasound transducer. In this case, no tissue was detected proximate the A and C ultrasound transducers in the near field scan. While the embodiment of FIG. 3E concerns the retrieval and display of previously collected information, the depth plot 333 (or any information of the chart 330 or otherwise referenced herein) can be presented in a live view showing information as it is collected. In this way, the tissue depth plot 333 can be presented live based on the information that is currently being sensed with the catheter 310.

Chart 330 includes an ablation monitoring plot 339 indicating the condition of an area of cardiac tissue as determined by ultrasound information, the area of cardiac tissue corresponding with a selected one of the markers 322. The ablation monitoring plot 339 indicates the degree to which the area of cardiac tissue was lesioned by an ablation therapy. The abscissa axis 336 of the plot 339 represents time (e.g., before, during, and after the delivery of the ablation therapy to a particular portion of cardiac tissue). The ordinate axis 335 represents the depth of the cardiac tissue. Specifically, shaded areas are represented to show tissue while unshaded areas of the plot 339 represent no cardiac tissue, such that the depth of the tissue is indicated by the height of the shaped area of the plot 339. The depth of the tissue can be determined based on the reflected ultrasound energy (e.g., a near field ultrasound scan performed according to A-mode or M-mode). The depth of cardiac tissue can be determined based on how long it takes ultrasound wave to be bounced back to the ultrasound transducer, where the longer it takes for the waves to reflected back to the ultrasound transducer the deeper the tissue reflecting the waves.

An ultrasound scan can characterize the state of tissue, and the different states of the tissue at different depths, which can be indicated in the ablation monitoring plot 339. For example, a first tissue state can be lesioned tissue and a second tissue state can be unlesioned tissue. In many cases, the ultrasound reflectivity properties of the tissue changes upon lesioning, so a change in the reflectivity can be used to determine whether tissue was lesioned. Before ablation therapy is delivered, a baseline assessment of the ultrasound reflectivity of an area of tissue can be determined. The baseline assessment can determine the intensity level of ultrasound energy reflected at various depths of the tissue. Baseline indicator 331 of chart 330 indicates the depth of the tissue for a particular area and further shows that the tissue has the same state (unlesioned) across the total depth of the tissue. The state of the tissue for the different depths can be indicated by different shading or coloring, however other manners of indicating different tissue states are also contemplated, such as labeling and/or numbering. The baseline indicator 331 can serve as a comparison, as it represents the state of the tissue area before any ablation therapy was delivered. The remainder of the ablation monitoring plot 339 (e.g., to the right of the baseline indicator 331, representing subsequently collected data) is based on the ultrasound information collected during the time that ablation therapy was being delivered. Unlesioned tissue indicator 337 (a lighter shade, also shown in the baseline indicator 331) indicates unlesioned (e.g., functioning) tissue at various depths while lesioned tissue indicator 338 (a darker shade) indicates tissue that has been lesioned. Depending on the type of ablation therapy, lesions may form on the surface of the cardiac tissue and then progress deeper as more ablation therapy is delivered. In some cases, lesions may form in the cardiac tissue and then progress deeper and toward the surface as more ablation therapy is delivered. The ablation monitoring plot 339 shows that over the time on the abscissa axis 336, the lesion penetrates deeper into the area of cardiac tissue. An ablation monitoring plot 339 can indicate that the lesion is transmural, such as by showing that the total depth of the tissue is indicated by lesioned tissue indicator 338 at a later point in time. To confirm the change to lesioned tissue, the ablation monitoring plot 339 can show that the tissue transitioned over time from unlesioned tissue indictor 337 in the baseline 331 to the lesioned tissue indicator 338 at a later time. However, the ablation monitoring plot 339 of FIG. 3E shows that the lesion was not transmural as the lesion does not cover the entire depth of the tissue for the particular area of tissue represented in the chart 330 (and associated with the marker selected by the curser 320). As such, this area could be a weak point in the conduction block and may necessitate further ablation therapy.

An indicator of the degree of which an ablation therapy lesioned tissue can be determined automatically based on reflected ultrasound or the changes in the speed or velocity of the sound waves. A threshold can be used to distinguish between lesioned and non-lesioned tissue for any of these characteristics. For example, a difference in a measure of ultrasound intensity (e.g., amplitude of an A-mode scan) between two different ultrasound scan times (e.g., a first baseline scan before ablation and a second scan during or after ablation) greater than a predetermined threshold can indicate that the tissue was lesioned. Control circuitry can automatically determine whether an area of tissue was lesioned based on determining whether the change in ultrasound intensity is greater than a predetermined threshold. A difference in ultrasound intensity less than the threshold can indicate an unsatisfactory lesion. A lack of change in ultrasound intensity can indicate no lesioning of the tissue at all. Such a determination can be performed for various depths of the same area of cardiac tissue (e.g., 0-1 mm depth, 1-2 mm depth, 2-3 mm depth, etc.) to assess the depth of lesioning.

It is noted that using reflected ultrasound information may be particularly useful for evaluating the efficacy of ablation therapy because the ultrasound information can indicate whether the tissue was lesioned and not just stunned, swollen, or otherwise temporarily electrically inactivated. An evaluation of the condition of ablated tissue based on electrophysiology can incorrectly identify tissue as lesioned when the aberrant electrical signal cannot be detected from the particular area of tissue. However, the tissue may not be fully lesioned and the electrical activity may return. Transmurally lesioned tissue, as determined by changes in intensity of reflected ultrasound energy, is less likely to later support the aberrant electrical activity and accordingly may be a more reliable indicator of durable lesioning.

Chart 330 includes a contact plot 332 indicating the orientation of the catheter at the time when the ablation therapy was delivered. Knowing the orientation of the catheter can be useful for determining how directly the ablation element was able to effectively deliver the ablation therapy based on collected ultrasound information. Contact plot 332 has a plurality of zones (A, B, C, and Tip) each corresponding to a respective ultrasound transducer orientated to face a different direction. For example, the A, B, and C ultrasound transducers can be arrayed around the circumference of the catheter 310 to cover different zones (e.g., each covering an arc of 120 degrees) around the 360 degree circumference of the catheter 310. The Tip ultrasound transducer can be positioned on the distal tip of the catheter 310 to point distally of the catheter 310. Each of the ultrasound transducers can operate with near-field functionality to detect tissue proximate the ultrasound transducer. In some cases, the system is configured to detect whether tissue is in contact with the catheter 310, which in many cases is the ideal position for delivering ablation therapy. The orientation of the catheter 310 can then be determined based on with which of the ultrasound transducers proximate or contacting tissue can be detected. The corresponding zone on the contact plot 332 can be highlighted to indicate with which of the ultrasound transducers proximate or contacting tissue can be detected. As shown in FIG. 3F, the Tip and B zones are highlighted, indicating that tissue is proximate these transducers, and the A and C zones are not highlighted, indicating that tissue is not proximate the ultrasound transducers that correspond to these zones. As such, at the time that the ultrasound information for a particular area of cardiac tissue was collected, the catheter 310 was orientated such that the Tip and B transducers were proximate cardiac tissue while the A and C transducers were not proximate tissue. This information can be used to determine how effectively the catheter 310 could have formed a lesion in the tissue. For example, if an ablation element generally directs ablation energy distally of the catheter, than whether the Tip zone is highlighted can indicate whether the catheter was optimally positioned to deliver an ablation therapy. If the contact plot 332 associated with a particular area of tissue indicates that the ablation element was not ideally positioned during the delivery, then the contact plot 332 might provide the basis for redelivering the ablation therapy to the tissue or closely monitoring the tissue for reversion back to supporting unwanted electrical activity.

In some cases, the contact plot 332 or other indicator of the orientation and proximity of the catheter to cardiac tissue can be used to determine how much ablation energy was delivered to an area of cardiac tissue. The size of a lesion, and the rate of growth of a lesion, are correlated with the amount of ablation energy delivered to an area of tissue. Therefore, an indicator of the proximity of the ablation element on the catheter to an area targeted for ablation can be factored into the degree to which the tissue was likely ablated by the therapy. Various variables can be integrated together to provide an indicator of the degree to which tissue was likely lesioned by delivery of ablation therapy. Such variables can include how much surface area of the ablation element is in contact with the targeted tissue, the power level of the ablation therapy, and/or the duration of the ablation therapy delivery to the area. These and/or other variables can be factored into an indicator of the extent to which an area of cardiac tissue was likely lesioned, the variables and/or the indicator then being displayed as part of the display of chart 330 and/or the map 300. For example, the size and/or color of each of the markers 322 on the map 300 can be based on such a factoring of these and/or other variables for each tissue area.

While the embodiment of FIG. 3E concerns the retrieval and display of previously collected information, the contact plot 332 (or any information of the chart 330 or otherwise referenced herein) can be presented in a live view showing information as it is collected. As such, the contact plot 332 can be displayed showing the orientation of the distal tip of the catheter relative to cardiac tissue in real time to facilitate navigation and ablation therapy delivery. The various sections of the contact plot 332 can be colored, shaded, or otherwise highlighted to show with which ultrasound transducers proximate or contacting tissue can be detected.

It is noted that the selection of the various markers 322 triggers the display of information, via chart 330, that was collected and saved in memory before the selection of the particular marker 322. As such, the markers 322 can represent a plurality of sets of selectively retrievable information. The information can be retrieved immediately following ablation delivery and/or at a much later time, such as weeks or months following ablation delivery. In this way, the markers 322 on the map 300 represent an interactive log of selectively retrievable information that can be reviewed to understand the state of the tissue. Understanding this information can be useful for determining how thorough an ablation therapy was performed and whether any weak points exist in a conduction block. For example, if an arrhythmia redevelops, this information can be reviewed to determine along which one or more areas are most likely to be supporting the unwanted electrical activity and/or identify areas for retreatment.

Although a chart 330 is displayed as an indicator of the degree of lesioning in response to the selection of a marker, other indications of the degree of lesioning can additionally or alternatively be displayed based on the selection. It will also be appreciated that different charts 330 can be retrieved and displayed depending on which one of the different markers 322 is selected. As such, a user can move from one marker to the next, a different chart 330 (or other information) being displayed for each of the marker selections, each of the different charts corresponding to the ultrasound information collected from that area of tissue that is associated with the selected marker.

FIG. 3E shows the selection of one of the markers 322 by moving the curser 320 to select the marker. However, markers and/or areas can be selected for ultrasound information retrieval and display in other manners. FIG. 3F shows chart 330 being displayed based on selection of an area of tissue associated with the information of the chart 330 based on the distal tip of the catheter 310 being moved to the area of cardiac tissue. As discussed herein, the mapping functions can determine the three dimensional position of the different areas of cardiac tissue and further the location of the catheter 310. As such, control circuitry can further compare this information to determine when the catheter (e.g., the tip of the catheter or the positional sensor within the catheter) is moved to one of the different areas of cardiac tissue. Based on the catheter 310 being moved to a particular area of cardiac tissue, ultrasound information collected from that particular area of cardiac tissue can be retrieved from memory and displayed. The ultrasound information may be displayed as a chart or other representation to indicate the degree to which the area of tissue was lesioned.

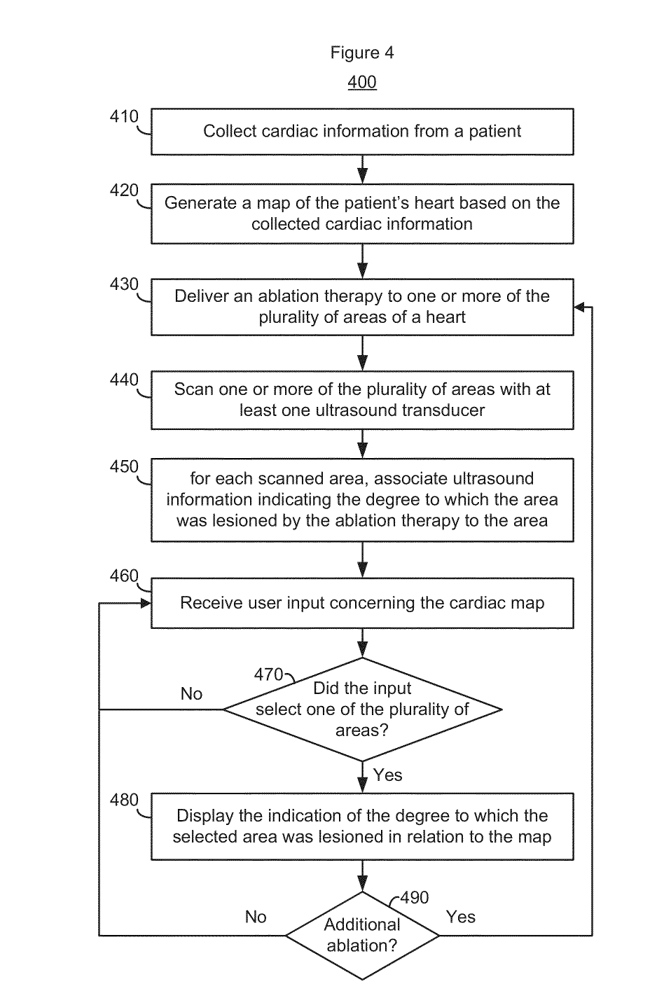

FIG. 4 illustrates a flow chart 400 of a method for representing tissue state information to assess and manage tissue ablation. The method includes collecting 410 cardiac information from a patient. The collection of 410 cardiac information can include sensing cardiac signals indicative of arrhythmia or other unwanted electrical cardiac activity as discussed herein. The collection of 410 information may include sensing location information indicating the three dimensional location of different areas of cardiac tissue as discussed herein.

The method further includes generating 420 a map of the patient's heart based on the collected 410 cardiac information. The map can be a two or three dimensional electroanatomical map indicating areas of aberrant electrical activity. Based on the map, or independently from the map, an ablation therapy can be delivered 430 to one or more of a plurality of areas of the heart. In various embodiments, ablation therapy will be delivered 430 to a plurality of different areas of cardiac tissue for at least a first iteration of the method (where one or more of the different areas can be retreated with ablation therapy as needed as further discussed herein). The plurality of areas of the heart can correspond to a contiguous line or other formation of tissue to form a block of electrical activation (e.g., to electrically isolate or destroy tissue activating out of synchrony with the rest of the cardiac tissue). The ablation therapy can be delivered 430 to the plurality of areas simultaneously or individually in series.

Before, during, and/or after the ablation therapy is delivered 430 to each area of cardiac tissue, each area to which ablation therapy is delivered 430 can be scanned 440 with an ultrasound transducer. In various embodiments, each area of tissue will be individually scanned 440 by an ultrasound transducer, where each area is scanned 440 separately (e.g., due to a limited field of view of near field ultrasound scanning). In various embodiments, a baseline level of ultrasound reflectivity can be determined for each area before ablation therapy is delivered to the area, and then one or more scans 440 of the same area can be performed during and/or after delivery 430 of the ablation therapy to the area. The ultrasound information collected in the scan 440 can be saved in memory. The information collected in the scan 440 can include, among other things, the level of ultrasound energy reflected from the area of cardiac tissue. In some cases, different levels of ultrasound energy reflected from the area of cardiac tissue can be measured and saved for different depths of the area of cardiac tissue.

For each scanned 440 area of cardiac tissue, ultrasound information collected from the area can be associated 450 with the area. Such associations 450 can be saved in memory to link the ultrasound information with the particular area of cardiac tissue, such that they can be retrieved together. The associated 450 information can include an indication of the degree to which the area of tissue was lesioned by the delivery 430 of the ablation therapy. The associated 450 information can include an indication of the level of ultrasound energy reflected from the area of cardiac tissue. The indicator of reflected ultrasound energy can be a portion of the ultrasound signal, a measure of the intensity of the ultrasound signal such as amplitude, a numerical value, and/or some other information derived from the ultrasound signal and indicative of a characteristic of the tissue from which the ultrasound waves reflected. Associating 450 can include determining that a particular portion of an ultrasound signal, indicative of the degree of lesioning, was sensed as reflected from the area of cardiac tissue. In some cases, the ultrasound signal is selectively sensed or portions of the signal are retained in memory based on correspondence to different areas of cardiac tissue.

The method further includes receiving 460 user input concerning the cardiac map. The user input can be any user input referenced herein (e.g., via a button, touch screen, touch pad, stylus, joystick, etc.) and can be an input selecting one of a plurality of markers on the map. The plurality of markers can respectively correspond to a plurality of areas of cardiac tissue to which ablation therapy was delivered 430. The plurality of areas and/or markers can be respectively associated 450 with ultrasound information. The received 460 input information can include moving a curser over the map, and the curser may be moved over one of the markers and/or areas to which ablation therapy was delivered 430. The method can include determining 470 whether the received 460 input selected one of the plurality of areas to which ablation therapy was delivered 430. If one of the areas is selected, then the indication of the degree to which the area was lesioned (the indication being associated 450 with the selected area), can be displayed 480 in relation to the map. For example, the indication can be displayed on the map. In some cases, the indication can be displayed alongside the map. If one of the areas is not selected (e.g., the curser is not over one of the ablated areas or a marker, or the catheter is not proximate one of the ablated areas) then the method can continue receiving 460 user input concerning the cardiac map until one of the areas is selected.

Based on the display 480 of the indication, a decision 490 can be made regarding whether further ablation is needed. For example, the indication may show that the degree of lesioning is not enough to assure that a reoccurrence of inappropriate conduction does not occur. One particular indication may show that a particular area has relatively less lesioning than the other indications displayed 480 for other areas of a conduction block, such that the particular area is a weak link in the conduction block and the most likely to support future reemergence of an arrhythmia. Ablation therapy may be redelivered 430 to one or more areas based on the indication, as guided by the selective display 480 of ultrasound information indicating the degree of lesioning.

As shown by the flow chart, further user input can be received 460. If it is determined 470 that the further input selects another one of the plurality of areas to which ablation therapy was delivered 430, then the currently displayed 480 indication can be replaced as the indication of the degree to which the newly selected area was lesioned is displayed 480 instead. In this way, information can be selectively displayed and replaced for the different areas as the areas are dynamically selected based on user input.

It is noted that various modifications can be made to the steps and/or the flowchart 400 of FIG. 4. In various embodiments, various steps of the method can be performed simultaneously or sequentially, such as delivering 430 the ablation therapy and scanning 440 the one or more areas with the ultrasound transducer. The order of the steps can be changed to any other order. In some cases, each of the steps of the method can be performed continuously or intermittently, for example, until no more inputs are received 460 or ablation is delivered 430. In some embodiments, collecting 410 the cardiac information, generating 420 the map, scanning 440 with ultrasound, associating 450, receiving 460 user input, and displaying 480 can be performed without ablation to profile the selected tissue area. For example, these steps, and/or any other steps referenced herein can be performed to assess the function of tissue without a preceding and/or subsequent ablation therapy being delivered. Such assessment may be to determine the state of cardiac tissue following infarction, arrhythmia (e.g., atrial fibrillation), or other event. The tissue being evaluated could be scar tissue created by a previous injury, fibrous tissue, tissue associated with myocardial infarction, or tissue subject to any event or condition that could potentially change the state of the tissue.

It is noted that the intensity of reflected ultrasound energy can change based on the distance between the ultrasound sensor and the tissue reflecting the ultrasound waves. Cardiac tissue is usually moving due to the constant dynamic function of the heart. Even inactivated cardiac tissue typically moves during a cardiac cycle and ultrasound energy measured from the tissue will change over a cardiac cycle. These changes could present themselves as changes in tissue characteristics (e.g., density), even if the state of the tissue does not change during the cardiac cycle. However, control circuitry can correct for the movement of tissue by various techniques. By monitoring tissue in an M-mode, dimensional and movement information can be collected. A signal indicative of the intensity of reflected ultrasound energy can be normalized in synchrony with the wall motion identified from an M-mode scan or the changes in the intensity of an ultrasound signal (e.g., the signal amplitude in A-mode) can otherwise be corrected or canceled out based on the wall motion known from the M-mode scan. In some embodiments, the distance between the ultrasound sensor and the tissue can be tracked by scanning in M-mode, and changes in the distance can be used to correct or cancel out changes in the signal intensity due to the distance changes. As such, various embodiments can include processing the signal containing the ultrasound intensity information to reduce or eliminate changes in the signal due to motion of the tissue relative to the sensor. Such processing can highlight changes in the signal due to changes in tissue characteristics indicative of a lesion.

In some embodiments, the repetitive motion of the plurality of areas of cardiac tissue can be detected in one or more ultrasound scans (e.g., in M-mode), and a map identifying the different areas (e.g., the map 300 of FIG. 3C) can represent the markers 322 to move to represent the repetitive cardiac motion. This information may be particularly useful to understand the continuity of a conduction block, where a series of ablated areas may be contiguous during one phase of the cardiac cycle but by stretching and/or contracting of the cardiac tissue, the tissue area may no longer be in a contiguous series in a different phase of the cardiac cycle. As such, markers corresponding to different areas of cardiac tissue can move in synchrony with the cardiac cycle to determine whether a break in lesion continuity between the areas of cardiac tissue is present over the cardiac cycle. If a break in lesion continuity is detected, the unlesioned area can be highlighted on the map. Ablation therapy can then be delivered to the area. The motion of the tissue can also be represented in relation to the catheter, and in particular the distal end of the catheter. Based on the changing distance between an area of targeted cardiac tissue and the distal end of the catheter, the delivery of ablation therapy can be timed over the cardiac cycle to when the targeted area is closest to the distal end of the catheter.

A characteristic of cardiac tissue that can indicate the degree of lesioning of an area of the tissue comprises the compression of the tissue over a cardiac cycle, where lesioned tissue does not compress during a cardiac cycle while non-lesioned or otherwise functioning tissue does contract over the cardiac cycle. The compressibility of tissue can be determined based on changes in density of the tissue over the cardiac cycle, where cardiac tissue typically becomes denser during the systolic phase and less dense during the diastolic phase. An area of cardiac tissue can be determined to be compressing when an indicator of tissue density (e.g., the level of intensity of received ultrasound energy) increases in a systolic phase and decreases in a diastolic phase. The different phases of the cardiac cycle can be determined based on an electrical cardiac signal (e.g., an electrocardiogram). Sections of tissue not fitting this profile can be determined to be lesioned. Sections of tissue fitting this profile can be determined to be functioning tissue and not lesioned, even if, for example, an electrical signal cannot be read directly from an electrode in contact with the tissue. The various embodiments of the present disclosure can indicate the degree of lesioning based on the compressibility of the area of cardiac tissue over a cardiac cycle. Determining the compressibility of cardiac tissue and other tissue characteristics, which can be applied to the methods and systems of the present disclosure, are further described in U.S. Provisional Patent Application No. 61/697,122, Filed Sep. 5, 2012 (Docket No. 432469.410146; 12-0080PV01), entitled CHARACTERIZATION OF TISSUE BY ULTRASOUND ECHOGRAPHY, which is expressly incorporated herein by reference in its entirety for all purposes.

It is noted that the steps of the method of FIG. 4, and/or any steps referenced herein, can be performed by control circuitry. For example, the steps of the method of FIG. 4 and/or any other steps referenced herein could be implemented by the system 100 of FIG. 1 in an automated manner by the control circuitry of FIG. 2. Likewise, any of the plots of FIGS. 3A-F and/or similar plots could be generated and displayed using the system 100 and control circuitry of FIG. 2 or any modifications thereof to characterize tissue and guide therapy.

The techniques described in this disclosure, including those of FIGS. 1-4 and those attributed to a system, control circuitry, processor, or various constituent components, may be implemented wholly or at least in part, in hardware, software, firmware or any combination thereof. A processor, as used herein, refers to any number and/or combination of a microprocessor, a digital signal processor (DSP), an application specific integrated circuit (ASIC), a field-programmable gate array (FPGA), microcontroller, discrete logic circuitry, processing chip, gate arrays, and/or any other equivalent integrated or discrete logic circuitry. “Control circuitry” as used herein refers to at least one of the foregoing logic circuitry as a processor, alone or in combination with other circuitry, such as memory or other physical medium for storing instructions, as needed to carry about specified functions (e.g., a processor and memory having stored program instructions executable by the processor for determining an indicator of the degree to which an area of cardiac tissue was lesioned by delivery of the ablation therapy based on an ultrasound signal, generating a map on a display representing the area, and representing the indication associated with the area when the area is selected on the map). The functions referenced herein may be embodied as firmware, hardware, software or any combination thereof as part of control circuitry specifically configured (e.g., with programming) to carry out those functions, such as in means for performing the functions referenced herein. The steps described herein may be performed by a single processing component or multiple processing components, the latter of which may be distributed amongst different coordinating devices. In this way, control circuitry may be distributed between multiple devices. In addition, any of the described units, modules, subsystems, or components may be implemented together or separately as discrete but interoperable logic devices of control circuitry. Depiction of different features as modules, subsystems, or units is intended to highlight different functional aspects and does not necessarily imply that such modules or units must be realized as hardware or software components and/or by a single device. Rather, specified functionality associated with one or more module, subsystem, or units, as part of control circuitry, may be performed by separate hardware or software components, or integrated within common or separate hardware or software components of control circuitry.

When implemented in software, the functionality ascribed to the systems, devices, and control circuitry described in this disclosure may be embodied as instructions on a physically embodied computer-readable medium such as RAM, ROM, NVRAM, EEPROM, FLASH memory, magnetic data storage media, optical data storage media, or the like, the medium being physically embodied in that it is not a carrier wave, as part of control circuitry. The instructions may be executed to support one or more aspects of the functionality described in this disclosure.

Although the embodiments referenced herein are described in the context of assessing the compressibility of cardiac tissue, the systems and methods referenced herein can be applied to profiling other areas of the body. For example, the systems and methods of this disclosure could be used for profiling or treating the prostate, brain, gall bladder, uterus, esophagus, and/or other regions in the body. Non compressing tissue can be identified as lesioned or otherwise non-functional tissue while compressing tissue can be identified as functioning tissue.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as falling within the scope of the claims, together with all equivalents thereof.

What is claimed is:

1. A system for characterizing the condition of multiple areas of cardiac tissue of a heart, the system comprising:
a catheter configured to be introduced into the heart, the catheter comprising:
at least one electrical sensor, the at least one electrical sensor configured to sense an electrical signal from the surface of the cardiac tissue;
at least one ultrasound transducer, the at least one ultrasound transducer configured to receive ultrasound energy reflected from the cardiac tissue and generate a signal indicative of the intensity of the reflected ultrasound energy; and
an ablation element, the ablation element configured to deliver an ablation therapy to the cardiac tissue;
a user interface comprising a display and a user input;
memory; and
control circuitry configured to determine, for each of a plurality of different areas of cardiac tissue, an indicator of the degree to which the area of cardiac tissue was lesioned by delivery of the ablation therapy based on the ultrasound signal, save the indicators for the plurality of different areas of cardiac tissue as respectively associated with the plurality of different areas of cardiac tissue in memory, generate a map on the display representing the plurality of different areas of the cardiac tissue based on the electrical signal, receive a selection of one or more of the plurality of different areas of the cardiac tissue from the user input, and represent the indication associated with each of the selected one or more areas of cardiac tissue on the map based on the selection.

2. The system of claim 1, wherein the indicators indicate the depth of lesioning through the cardiac tissue.

3. The system of claim 2, wherein the control circuitry is configured to determine the depth of lesioning through the cardiac tissue based on the intensity of ultrasound energy reflected by tissue of the area at different depths.

4. The system of claim 1, wherein the control circuitry is configured to determine, for each of the plurality of different areas, the degree to which the area was lesioned by calculating a change in a level of intensity of the ultrasound reflected from the area, the change calculated from a first ultrasound scan performed before delivery of the ablation therapy to the area and a second ultrasound scan performed following delivery of at least a portion of the ablation therapy to the area.

5. The system of claim 1, wherein the control circuitry is configured to represent the indication associated with each of the selected one or more areas of cardiac tissue by showing, on or alongside the map on the display, a graphic representation of tissue thickness and the depth of the lesion through the tissue thickness.

6. The system of claim 1, wherein the control circuitry is configured to represent the indicator associated with each of the selected one or more areas of cardiac tissue by showing, on or alongside the map on the display, a profile of ultrasound reflectivity of the area of cardiac tissue.

7. The system of claim 1, wherein the control circuitry is configured to represent the indication associated with each of the selected one or more areas of cardiac tissue in a manner that indicates whether the selected one or more areas of cardiac tissue was transmurally lesioned by the delivery of the ablation therapy.

8. The system of claim 1, wherein the control circuitry is configured to represent only one of the indicators of the degree to which the plurality of different areas of the cardiac tissue were lesioned at a time based on the user input.

9. The system of claim 1, wherein:
the at least one ultrasound transducer comprises at least three ultrasound transducers, the at least three ultrasound transducers positioned on different portions of the catheter to respectively scan different fields; and
the control circuitry is configured to determine the orientation of the catheter with respect to the cardiac tissue based on with which of the at least three ultrasound transducers the cardiac tissue can be detected to be proximate to the at least three ultrasound transducers and with which of the at least three ultrasound transducers the cardiac tissue cannot be detected to be proximate to the at least three ultrasound transducers.

10. The system of claim 8, wherein the control circuitry is configured to represent the orientation of the catheter in relationship to the map generated on the display.

11. The system of claim 1, wherein the control circuitry is configured to determine whether the plurality of different areas of cardiac tissue form a contiguous series and highlight one or more areas of the map where the contiguous series is not lesioned.

12. The system of claim 1, wherein:
the control circuitry is configured to move a curser generated on the display; and
the selection of the one or more of the plurality of different areas of the cardiac tissue is based on the curser being moved onto the one or more of the plurality of different areas on the map.

13. The system of claim 1, wherein:
the user input is controlled based on the movement of the catheter in the heart; and
the selection of the one or more of the plurality of different areas of the cardiac tissue is based on the catheter being moved onto the one or more of the plurality of different areas of the cardiac tissue in the heart.

14. The system of claim 1, further comprising a positional sensor on the catheter, the positional sensor configured to output a signal indicative of the spatial position of the catheter within the heart, wherein the control circuitry is configured to generate the map based on the signal and the electrical cardiac signal.

15. A method for representing information characterizing the condition of multiple areas of cardiac tissue of a heart, the method comprising:
sensing an electrical signal from the surface of the cardiac tissue with one or more electrodes on a catheter;
delivering an ablation therapy to a plurality of different areas of the cardiac tissue; for each of the plurality of different areas of the cardiac tissue, sensing an ultrasound signal with at least one ultrasound sensor within the heart, the ultrasound signal responsive to the ultrasound energy reflected from the area of cardiac tissue;
for each of the plurality of different areas of the cardiac tissue, associating with the area an indication of the degree to which the area of cardiac tissue was lesioned by the delivery of the ablation therapy based on the ultrasound signal;
representing a map of the plurality of different areas on a display, the map based at least in part of the electrical signal;
receiving a user input selecting one of the plurality of different areas; and
representing the indication associated with the selected one area based on the user input.

16. The method of claim 15, wherein the indications indicate the depth of lesioning through the cardiac tissue.

17. The method of claim 15, further comprising determining, for each of the plurality of different areas, the degree to which the area was lesioned by calculating a change in a level of intensity of the ultrasound reflected from the area, the change calculated from a first ultrasound scan performed before delivery of the ablation therapy to the area and a second ultrasound scan performed following delivery of at least a portion of the ablation therapy to the area.

18. The method of claim 15, wherein:
the at least one ultrasound sensor comprises at least three ultrasound sensors, the at least three ultrasound sensors positioned on different portions of the catheter to respectively scan different fields; and
the method further comprises determining the orientation of the catheter with respect to the cardiac tissue based on with which of the at least three ultrasound sensors the cardiac tissue can be detected to be proximate to the at least three ultrasound sensors and with which of the at least three ultrasound sensors the cardiac tissue cannot be detected to be proximate to the at least three ultrasound sensors.

19. A system for characterizing the condition of multiple areas of tissue, the system comprising:
a catheter, the catheter comprising:
at least one ultrasound sensor, the at least one ultrasound transducer configured to receive ultrasound energy reflected from the cardiac tissue and generate a signal indicative of the intensity of the reflected ultrasound energy; and
an ablation element on the catheter, the ablation element configured to deliver an ablation therapy to the cardiac tissue;
a user interface; and
control circuitry configure to determine, for each of a plurality of different areas of cardiac tissue, an indicator of the depth of lesioning through the cardiac tissue of the area by delivery of the ablation therapy based on the ultrasound signal and generate a map on the user interface representing the indicators of the depth of lesioning in respective association with the plurality of different areas of the cardiac tissue.

20. The system of claim 19, wherein the control circuitry is configured to determine the depth of lesioning, for each of the plurality of different areas of cardiac tissue, through the cardiac tissue based on the intensity of ultrasound energy reflected by cardiac tissue of the area at different depths.
